(19) United States
(12) Patent Application Publication
(10) Pub. No.: US 2002/0122795 A1
(43) Pub. Date: Sep. 5, 2002

(54) Compositions containing gangliosides for use in the treatment of skin disorders

(76) Inventor: Amy S. Paller, Wilmette, IL (US)

Correspondence Address: GARDNER, CARTON & DOUGLAS, 321 N. CLARK STREET, SUITE 3400, CHICAGO, IL, 60610, US

(21) Appl. No.: 10/013,788

(22) Filed: Dec. 7, 2001

Related U.S. Application Data
(60) Provisional application No. 60/254,387, filed on Dec. 8, 2000.

(51) Int. Cl. A61K 39/395; A61K 48/00; A61K 31/715

(52) U.S. Cl. 424/130.1; 514/44; 514/54

(57) ABSTRACT

The invention is a pharmacological composition that contains at least one ganglioside or portion thereof. The composition can be used to treat skin disorders.

### RELATED APPLICATION INFORMATION

This application claims priority from U.S. application Ser. No. 60/254,387 filed Dec. 8, 2000.

### GOVERNMENT RIGHTS

This invention was made with Government support under Grant No. AR-44619 awarded by the National Institutes of Health. The Government has certain rights in this invention.

### BACKGROUND OF THE INVENTION

Gangliosides are glycosphingolipids (carbohydrate and lipid-containing molecules) with sialic acid that are ubiquitous on the membranes of eukaryotic cells. Gangliosides have been implicated as regulators of cell growth and cellular interactions and can vary considerably in content during differentiation, ontogenesis and oncogenic transformations. Their role in the regulation of function of a variety of cells has been reported in the literature, including a report of increased G_{D3} on the surfaces of melanomas. The effect of gangliosides in skin cells, however, is not well known.

Disorders of the skin, such as cancers, psoriasis and ichthyosis, comprise a category of illnesses that is difficult to treat, due to the multiplicity of forms these diseases may take. In addition, many of the treatments for skin disorders involve toxic drugs, steroids or other substances that may have serious side effects or immunological incompatibilities. Because each disorder presents itself in a unique way in every individual, new therapies are continually needed, particularly therapies utilizing substances that are endogenous to the body.

Gangliosides and substances that control ganglioside content or activity (i.e., ganglioside modifiers) have been reported in the art, although reports of gangliosides in the skin or the use of gangliosides and ganglioside modifiers as therapeutic agents in the treatment of skin disorders are rare. We have discovered that certain gangliosides are present in skin cells, and that the levels of these gangliosides differ significantly in certain disorders from the same levels in normal skin cells.

For instance and as an example only, we previously discovered that GM3 was the predominant ganglioside in all skin cells, with smaller amounts of gangliosides GD3 and GT1b. Interestingly, we have noted increased 9-O-acetyl-GD3 in the epithelial tumors of patients with basal cells carcinomas, and increased 9-O-acetyl-GD3 has also been found in squamous cell cancers and in skin cells from patients with psoriasis. Our laboratory previously determined that gangliosides play an important role in skin cell function, including but not limited to the control of epidermal cell proliferation, differentiation, keratinocyte mobility and adhesion to matrices, and several other important processes localized to the skin.

We now disclose as part of the invention that certain gangliosides found in skin and their modifiers may be used as therapeutic agents in treating skin disorders, through a mechanism that involves modulation of ganglioside content in skin cells. Gangliosides are normal components of cells, and thus they are unlikely to raise any immunologic reaction. Moreover, it is believed that their non-steroidal, non-toxic nature could minimize the side effects present in many conventional treatments. Finally, modulation of ganglioside content is different from all other available therapies for skin disorders, including in the mechanism of action of gangliosides, suggesting that this therapy may be used in conjunction with other, currently available therapies.

### SUMMARY OF THE INVENTION

The invention is a pharmacological composition and methods of administration that are therapeutically effective in treating skin disorders. The composition contains at least one ganglioside or portion thereof. The ganglioside used in the composition can be synthetic or semi-synthetic and can contain a truncated lipid moiety. The truncated lipid moiety can contain from about 1 to about 17 carbons. In addition, the composition can also contain at least one agent that modulates ganglioside content or activity. Agents which can be used include, but are not limited to, one or more genes, antisense nucleic acids, anti-ganglioside antibodies or antibodies directed to ganglioside binding sites.

Thus, it is an object of the invention to provide a pharmacological composition comprising gangliosides or ganglioside modifiers for the treatment of skin disorders. It is another object of the invention to provide a method for the administration of the pharmacological composition.

### DETAILED DESCRIPTION OF THE PREFERRED EMBODIMENTS

The invention is a pharmacological composition for the treatment of skin conditions, such conditions including, but not limited to, skin cell overgrowth and undergrowth, wound healing, cell invasion, psoriasis and other inflammatory disorders, such as, but not limited to, allergic contact dermatitis and atopic dermatitis. The composition includes, but is not limited to, at least one ganglioside or portion thereof (including single fractions, binary mixtures or tertiary mixtures thereof). In addition, the composition can contain genes or other substances that modulate ganglioside content or activity, such as, but not limited to, anti-ganglioside antibodies or antibodies to ganglioside binding sites, antisense nucleic acids that bind to DNA or mRNA coding for genes that modulate ganglioside content or activity, or pharmacological agents that induce alterations in gangliosides or upregulate expression of their target molecules. In addition, the gangliosides used in the composition can be synthetic or semi-synthetic and contain a truncated lipid moiety having from about 1 to about 17 carbons. For example, an oligosaccharide can be synthesized and then linked with a shortened ceramide, such as a C2 or C8 ceramide, using routine techniques. Techniques for synthesizing gangliosides are known in the art (See Hideharu, I., et al., Trends in Glycoscience and Glycotechnology, 13(69):57-64 (2001), Ladisch, S., et al., Biochemistry, 34:1197-1202 (1995), Ladisch, S., et al., PNAS USA, 91:1974-1978 (1994), Ladisch, S. et al., Biochim. Phys. Acta, 1125:180-188 (1992) each incorporated by reference).

The compositions of this invention can be prepared in any suitable formulation now known or hereafter developed, including, but not limited to, ampoules, creams, ointments, gels, pellets, patches or solutions, in a pharmacologically acceptable carrier, which may optionally contain an adjuvant. The invention is administered to a patient by various suitable means now known or hereafter developed, including, but not limited to, topical delivery, subcutaneous or intralesional, intramuscular, transcutaneous and transdermal delivery, or gene therapy.

### Example 1

### Gangliosides as Modulators of Cell Growth

One manifestation of basal cell and squamous cell cancers of the skin, psoriasis, and genetic disorders of skin overgrowth, such as certain forms of ichthyosis, is excessive proliferation of skin. In our experiments, we have noted that alterations in ganglioside content, whether by pharmacologic manipulation or endogenous alteration through gene therapy, also affect the growth of skin cells. For instance, as an example and not as a limitation to the invention, we have determined that the pharmacologic addition of gangliosides of the “b” pathways of ganglioside synthesis and the precursor ganglioside, GM3, inhibit the proliferation of skin cells (See, Paller et al., J. Invest. Dermatol., 100:841-845 (1993) incorporated by reference). We have now determined that a decrease in the same gangliosides would increase the proliferation of skin cells; in fact, we have determined that transfection of a sialidase gene that cleaves the sialic acid group(s) from all gangliosides results in total ganglioside depletion, and leads to increased proliferation of skin cells. This increased proliferation is further accentuated by the addition of epidermal growth factor to the transfected cells in culture. We believe that a number of other gangliosides have similar effects, as described below, and could be used in therapeutic treatments.

The mechanism for the overgrowth of skin cells depleted of gangliosides and for the inhibition of growth of skin cells with increased ganglioside GM3 is decreased availability of the epidermal growth factor receptor for binding to its ligands (epidermal growth factor and transforming growth factor-α). This mechanism involves the direct binding of GM3 to the carbohydrate moieties of the epidermal growth factor receptor in keratinocytes. Also affected are downstream components in the epidermal growth factor signaling transduction pathway, e.g. GM3 downregulates the phosphorylation of MAP kinase and PI3 kinase.

These effects on cultured cells indicate that modulation of ganglioside content in vivo may lead to alterations in skin cell proliferation. Thus, increased content of gangliosides such as but not limited to GM3 in the skin could decrease the excessive proliferation of skin in disorders such as the common basal cell and squamous cell cancers of the skin, psoriasis, and genetic disorders of skin overgrowth such as ichthyosis. In addition, the cells in several neoplastic conditions have increased numbers of epidermal growth factor receptors, and increases in ganglioside content may be a means to modulate the activity of epidermal growth factor receptors and thus suppress the growth of neoplastic cells. In contrast, depletion of gangliosides may be a means to increase proliferation of keratinocytes, such as in condition of epidermal atrophy or to encourage wound healing.

In this invention, we utilize gangliosides and ganglioside modifiers to increase and decrease ganglioside content in skin cells, either by direct addition of gangliosides or portions thereof, or by modulation of enzymes that modify ganglioside synthesis pharmacologically or by gene introduction. For example, we have introduced different genes that encode enzymes that modulate ganglioside synthesis and metabolism into keratinocyte-derived cells. As a result, we have developed a wide array of transformed cells of the same origin that differ only in ganglioside expression, that is, cell lines that do not express gangliosides (sialidase gene), cell lines that have less GM3 and more GD3 (GD3 synthase gene), cell lines that have more GD2 and GT1b (GM2/GD2 synthase), and cell lines that have less GD3 but more 9-O-acetyl-GD3 (9-O-acetyltransferase). Selective overexpression of these various enzymes thus results in predictable and specific patterns of ganglioside alterations with specific effects on skin cell function. These cell lines will be useful for the study of the actions of specific gangliosides in skin cells in our laboratory and in the laboratories of other investigators. Also within the scope of the invention are antibodies that react to ganglioside binding sites or active sites, and nucleic acids that affect the expression of genes coding for gangliosides or other genes within the ganglioside regulation pathways.

### Example 2

### Gangliosides As Moderators of Fibronectin Matrix Interactions

Keratinocyte motility on a fibronectin substrate is critical in the reepithelialization of healing wounds, and in the spread of cutaneous malignancy. We discovered that gangliosides GT1b and GD3 inhibit the adhesion to and migration of keratinocytes and keratinocyte-derived cells on fibronectin (See, Paller, A. S., et al., J. Invest. Dermatol., 105:237-242 (1995); Sung, C-C., et al., Exp. Cell Res., 239:311-319 (1998); Wang, X-Q, et al., J. Biol. Chem., 276(48):44504-44511 (2001) each incorporated by reference). Conversely, endogenous depletion of gangliosides through sialidase gene modulation leads to increased adhesion of the cells to fibronectin and vitronection. Gangliosides also trigger the apoptosis of keratinocytes when plated on a fibronectin matrix.

The mechanism of the ganglioside effect involves the direct interaction of keratinocyte a5b1 with GT1b (and GD3), resulting in inhibition of phosphorylation of FAK and also of integrin-linked kinase. The ganglioside specifically interacts through carbohydrate moieties, with preference for the a5 subunit of the a5b1. Integrin avb3, which is structurally similar to a5b1, binds to GD3 and GD2, which limit the adhesion to vitronectin. Although any interaction between ganglioside and integrin b1 is much weaker, GT1b is able to bind b1 and, by doing so, inhibits b1 phosphorylation and serine/threonine phosphorylation.

The interaction of a5b1 and fibronectin is considered critical for embryonic development of skin, healing of wounds and spread of cutaneous malignancies. In addition, it is known in the art that cells from patients with psoriasis have shown increased adherence to fibronectin and a decreased ability to undergo apoptosis. We have confirmed that explant cultures from patients with psoriasis respond to pharmacological therapy with GT1b with clear inhibition of attachment, just as in normal cells, suggesting clinical efficacy in psoriasis. Integrin avb3 plays a vital role in the development and proliferation of blood vessels in skin; increased activity leads to neovascularization, while inhibition can inhibit new vessel formation. Thus, an increase in more complex skin gangliosides, particularly GT1b, may be novel agents for treating psoriasis or in inhibiting vascular-dependent processes, such as the rapid growth of hemangiomas in infants. In contrast, depletion of gangliosides (e.g., through increased local sialidase activity) may encourage wound healing, through increasing the migration of keratinocytes, increasing keratinocyte proliferation, and encouraging angiogenesis. Thus, the invention includes administering gangliosides including but not limited to GT1b and GD3, and other agents that affect the amount and action of these gangliosides, to control fibronectin interactions.

### Example 3

### The Use of Gangliosides to Regulate Matrix Metalloproteinase (MMP) Activity

Interestingly, we have discovered that gangliosides have other therapeutic effects as well, such as an effect on matrix metalloproteinase activity (See, Wong, A-Q., et al., J. Invest. Dermatol., 114:8-12 (2000) incorporated by reference). Sialidase-transfected keratinocytes with ganglioside depletion showed markedly increased levels of metalloproteinase, namely TIMP-1, resulting in significantly decreased activity of MMP-9. In contrast, pharmacologic addition to normal keratinocyte-derived cells of GM3 or GT1b results in a marked suppression of expression of both TIMP-1 and MMP-9 expression, without affecting the activity of MMP-9. We have also determined that addition of epidermal growth factor (EGF) further increased the expression and activity of MMP-9 expression, without affecting the activity of MMP-9, and increases its activity in the SCC12 cells, but does not affect the expression of TIMP-1. In contrast, inhibitors of protein kinase C (PKC) decrease the expression of TIMP-1 in SCC12 cells without altering MMP-9 expression. These data suggest that gangliosides modulate the expression of MMP-9 and TIMP-1 through their effects on epidermal growth factor receptor function and protein kinase C function, respectively. In other studies, GT1b, GD3 and, to a lesser extent, GM3 inhibit the expression and activity of MMP-2. In contrast, activators of PKC activity (including but not limited to concanavalin A and PIP₃) increased MMP-2 activity and expression by the keratinocyte-derived SCC12 cells, and only slightly increase TIMP-2 expression. When both activators of PKC and gangliosides GT1b or GD3 are incubated together with the SCC12 cells, no change in the expression or activity of MMP-2 is noted. These studies provide evidence that more highly sialylated gangliosides modulate MMP-2 expression and activity in keratinocytes through a mechanism that involves suppression of the PKC signaling pathway.

We also believe that, since MMP-9 and MMP-2 are both important for spread of cutaneous malignancy, the invention's methods of modulation of MMP activity may affect the ability of skin cancers to spread, and thus this invention also represents a new non-surgical intervention. The importance of matrix metalloproteinase activity in angiogenesis also suggests roles for gangliosides through different mechanisms on neoplastic growth (including but not limited to melanoma and hemangiomas in babies), wound healing, hair growth and embryologic skin development.

Thus, the invention includes the use of gangliosides, including but not limited to GM3, GT1b, 9-O-acetyl-GD3, and GD3, and modifiers of these gangliosides or their activity, to regulate matrix metalloproteinase activity and provide a therapeutic treatment for related skin disorders.

### Example 4

### In vivo Studies

In vivo studies have been performed in which cells transfected with genes that encode enzymes that modify synthesis in metabolism have been injected into the skin of immunodeficient mice and the resultant tumors characterized. These cells in vitro have shown specific alterations in gangliosides as predicted for the specific enzyme overexpressed. The in vitro characteristics of the cells are maintained and manifest in vivo in the tumors grown in these mice. Tumors with parental or vector transfected control cells rarely if ever are produced and are small and well-differentiated in appearance. Similarly, cells transfected with GM2/GD2 synthase produce small tumors that resemble those of the parental cells. In contrast, the tumors from cells transfected with genes that express sialidase, GD3 synthase, or 9-O-acetyltransferase grew quickly and to a large size; although characteristics among the hyperproliferative tumor groups differed, the sialidase overexpressing tumors in particular shows poor differentiation and rapid growth with a malignant, “aneuploid” phenotype and huge numbers of mitotic cells.

The foregoing are offered for purposes of illustration and example. It is intended that certain variations in the components, proportions, substances, ingredients, methods of administration and other parameters of the invention described herein will be obvious to one skilled in the art, and that such variations are within the scope of this invention.

What is claimed is:

1. A pharmacological composition comprising at least one ganglioside or portion thereof; wherein the pharmacological composition modulates the content of the ganglioside in the skin.

2. The pharmacological composition of claim 1 wherein the ganglioside is GM3, GT1b, 9-O-acetyl-GD3 or GD3.

3. The pharmacological composition of claim 1 wherein the ganglioside is a synthetic or semi-synthetic ganglioside having a truncated lipid moiety.

4. The pharmaceutical composition of claim 3 wherein the truncated lipid moiety contains from about 1 to about 17 carbons.

5. The pharmacological composition of claim 1 further comprising at least one agent that modulates ganglioside content or activity.

6. The pharmacological composition of claim 5 wherein said agent is a gene, an antisense nucleic acid, anti-ganglioside antibodies or antibodies directed to ganglioside binding sites.

7. The pharmacological composition of claim 1 further comprising a pharmacologically acceptable carrier.
